(19) United States
(12) Patent Application Publication
(10) Pub. No.: US 2018/0333590 A1
(43) Pub. Date: Nov. 22, 2018

(54) HEAD WORN DEVICE FOR TREATING ALZHEIMER'S DISEASE

(71) Applicants: Matthew D. Millard, Cambridge, MA (US); David A. Gonzales, Lakewood, CO (US)

(72) Inventors: Matthew D. Millard, Cambridge, MA (US); David A. Gonzales, Lakewood, CO (US)

(21) Appl. No.: 15/597,520

(22) Filed: May 17, 2017

(51) Int. Cl. A61N 5/06

(52) U.S. Cl. CPC A61N 5/0618; G02C 11/10

(57) ABSTRACT

A head worn device for treating Alzheimer's disease. The head worn device has LEDs mounted to the device. A printed circuit board (PCB) is also mounted on the head worn device. The PCB has a microprocessor and a battery in electrical communication. The LEDs are in also in electrical communication with the microprocessor. The microprocessor is programmed to control the flashing of the LEDs so that they flash at a regulated frequency. The flashing LED light enters the eyes of the user and travels to the visual cortex for Alzheimer's treatment. In a preferred embodiment the LEDs flash at a gamma frequency.

The present invention relates devices for treating diseases, and in particular, devices for treating Alzheimer's disease.

### BACKGROUND OF THE INVENTION

Alzheimer's dementia is a horrible disease that affects many people. Approximately 5.5 million Americans are currently living with Alzheimer's in 2017. One in ten people age 65 and older currently has Alzheimer's.

Traditionally, Alzheimer's research has focused on the impact of genetics on the disease. In recent years, however, focus has shifted to treating the brain itself. It is known that neurons in the brain interact with each other and will fire at various frequencies. Gamma frequency, defined as the frequency range of 30 Hz to 100 Hz, is important for higher order cognitive function. It has been widely recognized that Alzheimer's patients have diminished neuron activity, especially with respect to the gamma frequency range. Alzheimer's patients also have elevated levels of beta-amyloid peptides. The beta-amyloid peptides are proteins that hinder and block neuron signals, including the gamma oscillation.

At Massachusetts Institute of Technology (MIT) experimentation has been conducted and published that has shown positive results gained after treating mice affected with Alzheimer's. For example, mice suffering with Alzheimer's were exposed to light from Light Emitting Diodes (LEDs) flashing at a gamma frequency of 40 Hz. The mice were placed in a dark area and exposed to a specific frequency light oscillation from LEDs in close proximity. The mice could see the flashing light and it entered their brains through the visual cortex. The visual cortex of the brain is a part of the cerebral cortex that plays an important role in processing visual information.

The treated mice showed remarkable improvement. For example, after an hour of stimulation at 40 Hz, the researchers found a 40 to 50 percent reduction in the levels of beta amyloid proteins in the hippocampus. Additionally, the light exposure stimulated microglia cells. Microglia functions to help remove beta amyloid proteins.

In summary, directly exposing parts of a mouse brain to gamma oscillations supports at least two pathways that aid in the treatment of Alzheimer's. One is to reduce beta amyloid production from neurons. The second is to enhance the removal of beta amyloid by microglia.

More detailed discussions of recent advancements with gamma frequency exposure to Alzheimer infected mice are discussed in the following publications available via the Internet at the following website addresses:
1) http://www.nature.com/nature/journal/v540/n7632/abs/nature20587.html
2) http://www.radiolab.org/story/bringing-gamma-back/3)
http://news.mit.edu/2016/visual-stimulation-treatment-alzheimer-1207
4) http://www.latimes.com/science/sciencenow/la-sci-sn-led-lights-alzheimers-plaques-20161206-story.html

There has not been significant study of the effect of gamma frequency light stimulation on humans. However, the studies on mice lend credence to the hypothesis that a human brain will function similarly to the brain of a mouse and that gamma frequency exposure will reduce beta amyloid production from neurons and enhance the clearance of beta amyloid by microglia. Nevertheless, for the successful treatment of a human, there must be a safe, comfortable, dignified and humane way of providing treatment.

What is needed is an effective way to treat or reduce the effects of Alzheimer's disease in patients by delivering light to the patient that is oscillating in the gamma frequency.

### SUMMARY OF THE INVENTION

The present invention provides a head worn device for treating Alzheimer's disease. The head worn device has LEDs mounted to the device. A printed circuit board (PCB) is also mounted on the head worn device. The PCB has a microprocessor and a battery in electrical communication. The LEDs are also in electrical communication with the microprocessor. The microprocessor is programmed to control the flashing of the LEDs so that they flash at a regulated frequency. The flashing LED light enters the eyes of the user and travels to the visual cortex for Alzheimer's treatment. In a preferred embodiment the LEDs flash at a gamma frequency.

### BRIEF DESCRIPTION OF THE DRAWINGS

FIGS. 1 and 3 shows a preferred embodiment of the present invention

FIG. 2 shows a preferred speaker ear piece.

FIG. 4 shows a user wearing a preferred embodiment of the present invention.

FIG. 5 shows another preferred embodiment of the present invention.

FIG. 6 shows another preferred embodiment of the present invention.

FIG. 7 shows another preferred embodiment of the present invention.

FIG. 8 shows another preferred embodiment of the present invention.

FIG. 9 shows another preferred embodiment of the present invention.

### DETAILED DESCRIPTION OF THE PREFERRED EMBODIMENTS

In preferred embodiments of the present invention LEDs are mounted on eyeglass frames so that the LEDs are in close proximity to the user. The LEDs are programmed to flash at a gamma frequency of 30-100 Hz. For example, in one preferred embodiment the LEDs flash at 40 Hz. The user is therefore able to easily be exposed to the flashing light. The light enters the user's brain through the eyes and travels to the visual cortex. The user's brain is then exposed in a manner similar to that discussed above allowing the brain treatment for Alzheimer's by reducing beta amyloid production from neurons and enhancing the clearance of beta amyloid by microglia.

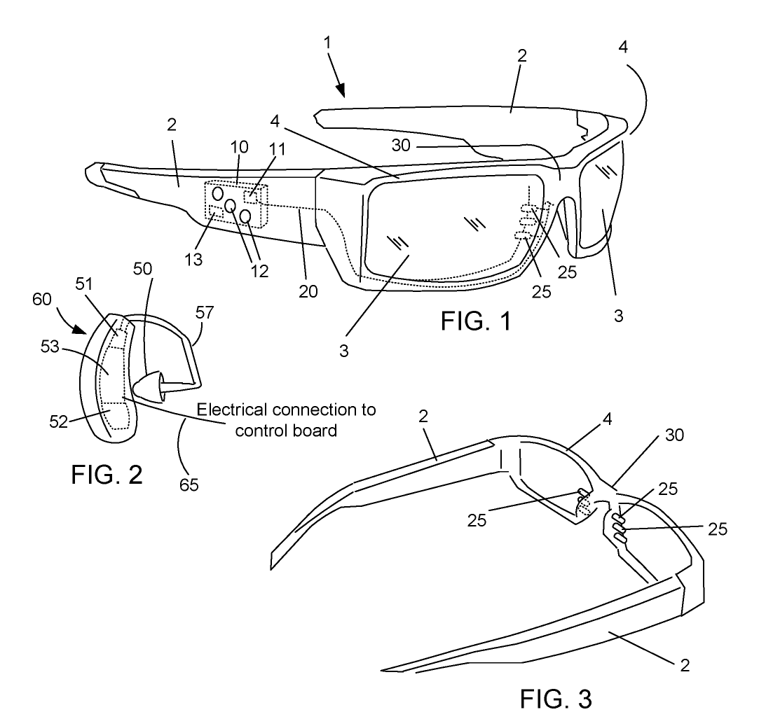

FIG. 1 shows a first preferred embodiment of the present invention. Glasses 1 include temples 2, lenses 3, and lens frame 4. Printed circuit board 10 is mounted into temple 2 as shown. PCB 10 includes microprocessor 11, control switches 12 and battery 13, each of which is in electrical communication with one another. Electric wire 20 extends from PCB 10 and runs through temples 2 and frames 4 and connects microprocessor 11 to LEDs 25, as shown. It should be noted that in another preferred embodiment control switches 12 can be replaced with a remote control device.

Microprocessor 11 is programmed to control the flashing rate of LEDs 25 so that they oscillate at 40 Hz or in the gamma frequency range. In a preferred embodiment, the user can turn LEDs 25 on or off by using control switches 12. Also in a preferred embodiment the user can vary the oscillation rate of LEDs 25 with switches 12. In a preferred embodiment LEDs 25 will always oscillate within the gamma frequency range, at a value between 30 Hz and 100 Hz.

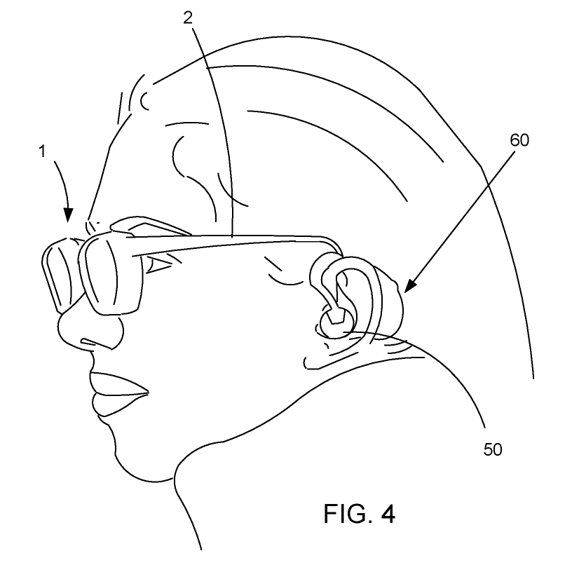

As shown in FIGS. 1 and 3, LEDs 25 are mounted on frame 4 near nose support 30. However, it should be noted that LEDs 25 may be mounted anywhere on frame 4 behind lenses 3 so that flashing light from LEDs 25 is able to enter the eyes of the user and travel to the visual cortex for Alzheimer's treatment.

In another preferred embodiment, ear mold 50 is inserted into a user's ear. Speaker earpiece 60 includes battery 52, amplifier 53 and speaker 51. Amplifier 53 is in electrical communication with PCB 10 and microprocessor 11 via wire 65. Ear mold 50 receives audio from speaker 51 via tubing 57. In a preferred embodiment, ear mold 50 transmits oscillating audio to the user that pulsates at the gamma frequency, preferably 40 Hz, or other frequency. The audio is heard by the user through the ear, traveling to the user's brain. In a preferred embodiment, the audio is preferably in sync with the flashing light at 40 Hz. FIG. 4 shows a side view of a user donning glasses 1 also using speaker earpiece 60 with ear mold 50 inserted into her ear.

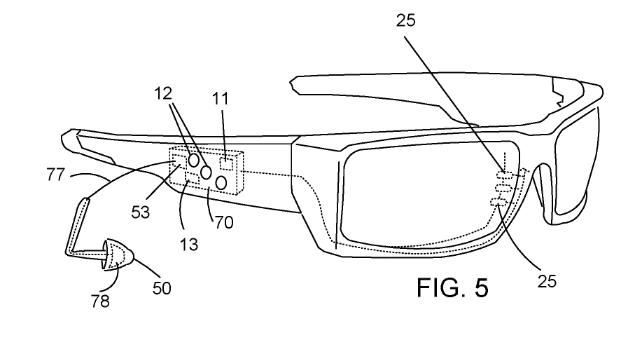

FIG. 5 shows another preferred embodiment of the present invention. PCB 70 includes microprocessor 11, control switches 12 and battery 13, each of which is in electrical communication with one another. PCB 70 also includes amplifier 53. Speaker wire 77 connects amplifier 53 to speaker 78 in ear mold 50. In a preferred embodiment, ear mold 50 transmits oscillating audio to the user that pulsates, preferably in the gamma frequency, or more specifically at 40 Hz. The audio is heard by the user through his ear and travels to the user's brain. The audio is preferably in sync with the flashing light from LEDs 25 at 40 Hz.

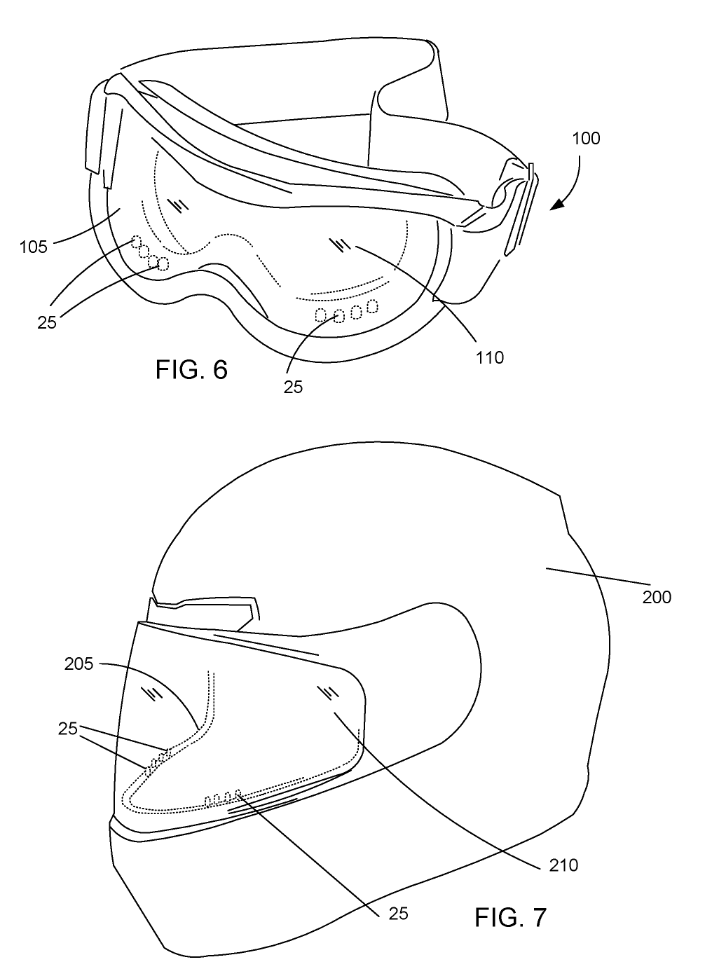

The above preferred embodiments showed LEDs 25 mounted on eye glasses. Eye glasses are comfortable to wear and can be worn with dignity and ease. It also should be noted that there are other types of head worn devices that may also be utilized with similar effectiveness. For example, FIG. 6 shows LEDs 25 mounted on goggle frame 105 of goggles 100 behind lens 110. As with eye glasses 1, flashing light from LEDs 25 is able to enter the eyes of the user and travel to the visual cortex for Alzheimer's treatment. Also, FIG. 7 shows LEDs 25 mounted on helmet frame 205 of helmet 200 behind lens 210. As with eye glasses 1, flashing light from LEDs 25 is able to enter the eyes of the user and travel to the visual cortex for Alzheimer's treatment.

### Other Preferred Embodiment

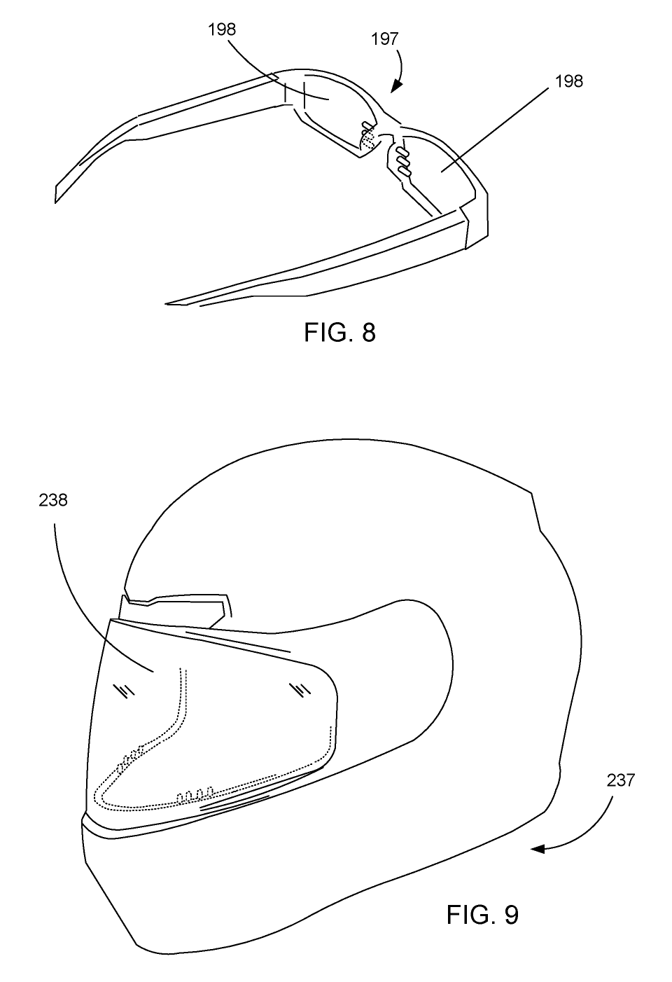

Above it was explained that lenses 3 allow a user to see through eye glasses 1 while being treated. In another preferred embodiment lenses are omitted and instead the user's eyes are covered by an opaque covering. For example, FIG. 8 shows head worn device 197 having opaque lenses 198. Also, FIG. 9 shows head worn device 237 having opaque lens 238. It would also be possible to utilize a head worn device in the shape of a box as well, having no lenses and with the LEDs attached directly to the head worn device.

Although the above-preferred embodiments have been described with specificity, persons skilled in this art will recognize that many changes to the specific embodiments disclosed above could be made without departing from the spirit of the invention. For example, even though the above preferred embodiments discussed LEDs flashing at 40 Hz, it would be possible to adjust the frequency of the flashing to a different value, preferably in the gamma frequency range of 30 to 100 Hz. In another preferred embodiment the LEDS are programmed to flash at a frequency outside the gamma frequency range. Therefore, the attached claims and their legal equivalents should determine the scope of the invention.

What is claimed is:

1. A head worn device for treating Alzheimer's disease, comprising:
A. a plurality of LEDs mounted to said head worn device,
B. a PCB mounted on said head worn device, said PCB comprising:
a. a microprocessor,
b. a battery, wherein said microprocessor said battery are each in electrical communication, wherein said plurality of LEDs are in electrical communication with said microprocessor,
wherein said plurality of LEDs are programmed to flash at a regulated frequency so that said flashing light enters the eyes of the user and travels to the visual cortex for Alzheimer's treatment.

2. The head worn device as in claim 1, wherein said regulated frequency is a gamma frequency.

3. The head worn device as in claim 2, further comprising:
A. a lens to permit viewing, and
B. a frame for supporting said lens, wherein said plurality of LEDs is mounted to said frame.

4. The head worn device as in claim 2, further comprising a control switch in electrical communication with said microprocessor for controlling the flashing of said plurality of LEDs.

5. The head worn device as in claim 2, further comprising a remote control device in electrical communication with said microprocessor for controlling the flashing of said plurality of LEDs.

6. The head worn device as in claim 2, wherein said gamma frequency is 40 Hz.

7. The head worn device as in claim 2, wherein said head worn device is eye glasses comprising temples connected to said frame, wherein said PCB is mounted into said temples.

8. The head worn device as in claim 2, further comprising an ear mold having a speaker that generates audio frequencies at gamma frequency,

9. The head worn device as in claim 8, wherein said gamma frequency is 40 Hz.

10. The head worn device as in claim 2, wherein said head worn device is goggles.

11. The head worn device as in claim 2, wherein said head worn device is a helmet.

12. A head worn device for treating Alzheimer's disease, comprising:
A. a lens to permit viewing,
B. a frame for supporting said lens,
C. a plurality of LEDs mounted to said frame,
D. a PCB mounted on said head worn device, said PCB comprising:
a. a microprocessor,
b. a battery, wherein said microprocessor said battery are each in electrical communication, wherein said plurality of LEDs are in electrical communication with said microprocessor,
wherein said plurality of LEDs are programmed to flash at a gamma frequency so that said flashing light enters the eyes of the user and travels to the visual cortex for Alzheimer's treatment.
